(19) United States
(12) Patent Application Publication
(10) Pub. No.: US 2016/0348155 A1
(43) Pub. Date: Dec. 1, 2016

(54) METHOD FOR EVALUATING THE EFFECTS OF A COMPOSITION COMPRISING MICROORGANISMS ON INTESTINAL MICROBIOTA

(71) Applicant: SOFAR S.P.A., Trezzano Rosa (milano) (IT)

(72) Inventors: Simone Domenico GUGLIELMETTI, MILANO (IT); Ruggero ROSSI, MILANO (IT); Walter FIORE, TREZZANO ROSA (IT); Andrea BIFFI, URGNANO (IT)

(21) Appl. No.: 14/916,959

(22) PCT Filed: Sep. 5, 2014
(86) PCT No.: PCT/IB2014/064284

§ 371 Date: Mar. 4, 2016

(30) Foreign Application Priority Data
Sep. 6, 2013 (IT) MI2013A001473

(51) Int. Cl. C12Q 1/68

(52) U.S. Cl. CPC C12Q 1/689; C12Q 2600/158; C12Q 2600/136

(57) ABSTRACT

The present invention relates to a method for determining the probiotic/paraprobiotic activity of a composition comprising microorganisms, in particular bacteria, said method being based on evaluating, by metagenomic analysis, the qualitative and/or quantitative change in faecal microbiota following intake of the composition. Moreover, the present invention relates to a kit for carrying out said method.

The present invention relates to a method for determining the probiotic/paraprobiotic activity of a composition comprising microorganisms, in particular bacteria, said method being based on an evaluation of the qualitative and/or quantitative change in faecal microbiota following intake of the composition. Moreover, the present invention relates to a kit for carrying out said method.

The gastrointestinal tract comprises numerous populations of microorganisms which have developed and multiplied during the development of each individual and form the so-called intestinal microbiota or intestinal flora.

Therefore, the intestinal microbiota represents a highly complex ecosystem and the condition of equilibrium among the different populations of microorganisms making it up, or so-called eubiosis, is fundamental in order to ensure the body's well-being and health, since the microbiota significantly conditions the development and the homeostasis of the intestinal mucosa of the host individual.

In other words, the intestinal microbiota represents a veritable organ. In fact, qualitative and/or quantitative modifications in the intestinal microbiota of an individual, or so-called disbiosis or dismicrobism, can result in the loss of the intestinal homeostasis, which in turn can condition the etiopathogenesis of a broad spectrum of pathologies.

For the purpose of treating a condition of intestinal disbiosis, or in any case for the purpose of maintaining the equilibrium of the intestinal microbiota, the practice of taking probiotic/paraprobiotic products is becoming more and more frequent.

According to the definition of the FAO/WHO, a probiotic is a set of “live microorganisms which, when administered in adequate amounts, confer a health benefit on the host”.

In light of the above, the advantages tied to the development of a method making it possible to evaluate, quickly and reliably, the effects of an exogenous composition/formulation comprising microorganisms on the bacterial composition of the intestinal microbiota of an individual are fairly evident.

In fact, on the basis of the effects measured with such a method, i.e. on the basis of how the intake of the composition comprising microorganisms quantitatively and/or qualitatively modifies the intestinal microbiota, it will be possible to establish whether said composition is capable of favouring and/or ensuring the well-being and health of the human body and, therefore, whether it fulfils one of the fundamental prerequisites for being identified as a probiotic/paraprobiotic.

The present invention fulfills the above-mentioned requirements by providing a method for determining, by molecular analysis, the qualitative and/or quantitative change in the composition of the faecal microbiota of an individual following intake of a composition comprising microorganisms, preferably bacteria, according to a randomized, double-blind, placebo-controlled crossover protocol.

In fact, the Applicant has experimentally demonstrated, for the very first time, the necessity of conducting crossover intervention study protocols, especially on a healthy population, in order to prevent the marked inter-individual variability from hiding the possible effects of a treatment, in particular a treatment with a probiotic/paraprobiotic, or from leading to false statistical positives.

The method of the present invention, besides being particularly advantageous for the purpose of determining the effects of a generic composition comprising microorganisms (i.e. a presumed probiotic/paraprobiotic) on faecal microbiota, is also useful for the purpose of confirming the health-promoting effect of a known probiotic/paraprobiotic on the human body, or for the purpose of determining any new specific effects of a known probiotic/paraprobiotic, for example by studying which populations of microorganisms are stimulated and/or inhibited in their growth following intake of the composition. In fact, on the basis of the main activities in which the populations of microorganisms whose growth is stimulated and/or inhibited following intake of the composition are involved, it will be possible to define the possible new effects of the same. For example, if, following intake of a probiotic according to the method of the present invention, it is found that a particular bacterial population has grown in quantitative terms and that this bacterial population has a metabolism mainly involved in the production, for example of butyric acid, it can be deduced that the probiotic can be taken in order to increase the amount of butyric acid in the intestinal tract.

Further advantages of the method of the present invention will be more apparent from the detailed description that follows and from the examples, which, however, have only a demonstrative, non-limiting purpose.

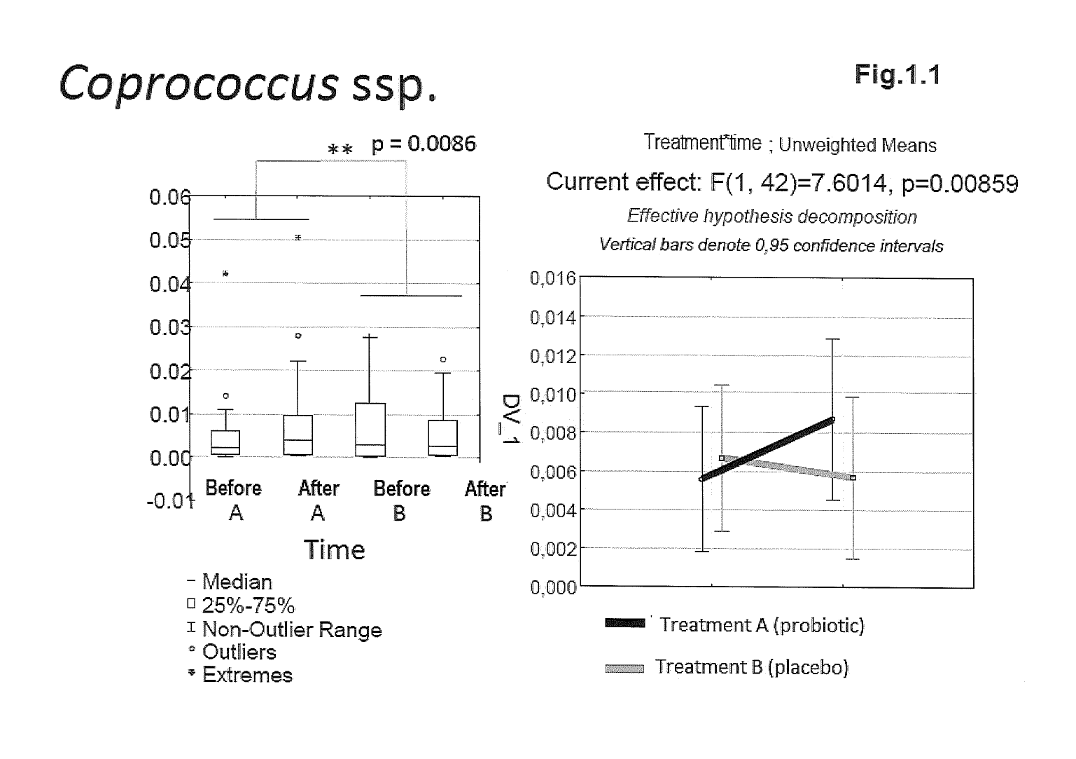

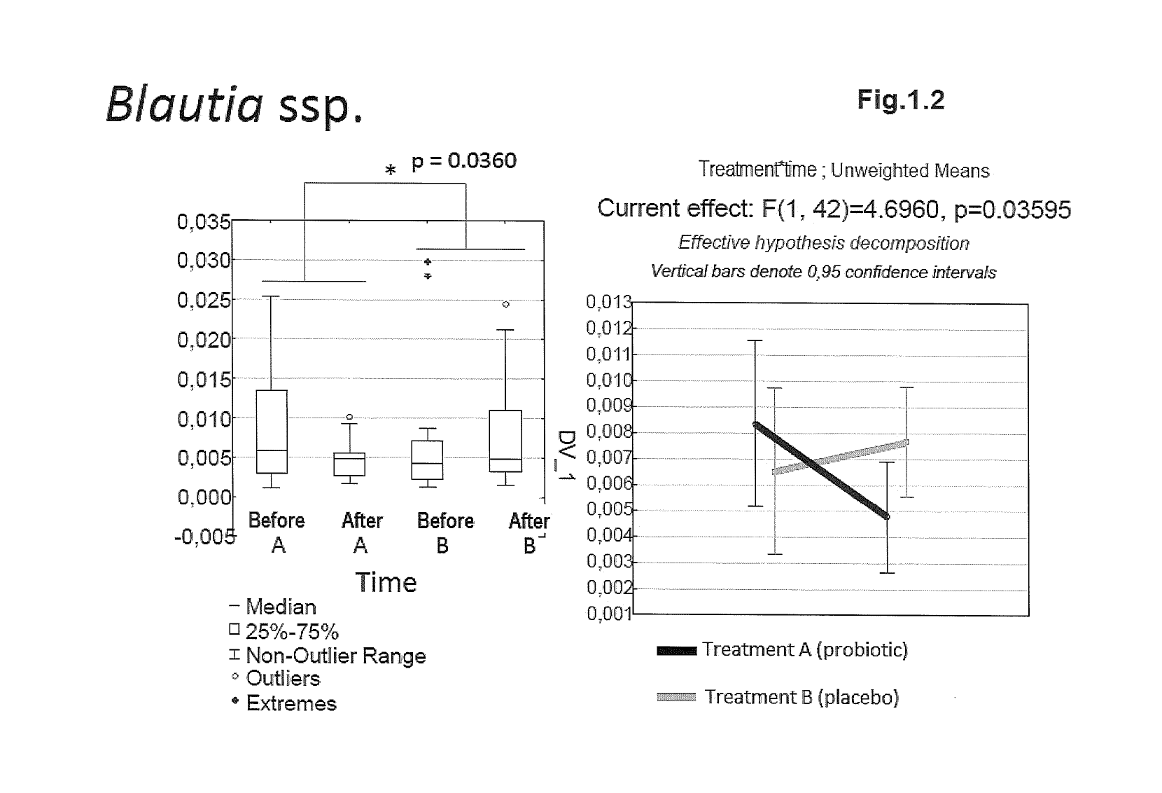

To enable a better understanding of the detailed description, FIGS. 1-4 have been appended hereto:

FIG. 1 shows the result of the statistical analysis conducted in order to evaluate the increase in the population of bacteria of the genus Coprococcus (FIG. 1.1) and the decrease in the population of bacteria of the genus Blautia (FIG. 1.2) before and after treatment with the composition of the present invention (A) and, at same time, the decrease in the population of bacteria of the genus Coprococcus (FIG. 1.1) and the increase in the population of bacteria of the genus Blautia (FIG. 1.2) before and after treatment with the placebo (B);

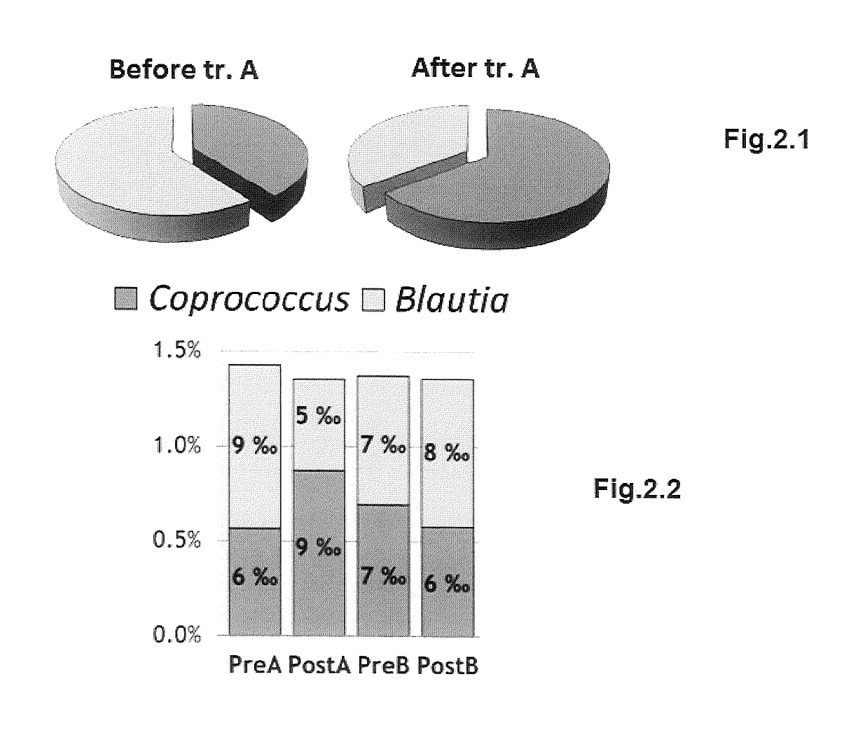

FIG. 2.1 shows the increase in the population of bacteria of the genus Coprococcus (dark grey) and the decrease in the population of bacteria of the genus Blautia (light grey) before and after treatment with the composition of the present invention;

FIG. 2.2 shows the percentage increase in the population of bacteria of the genus Coprococcus (dark grey) and the percentage decrease in the population of bacteria of the genus Blautia (light grey) before and after treatment with the composition of the present invention (A) and the percentage decrease in the population of bacteria of the genus Coprococcus (dark grey) and the percentage increase in the population of bacteria of the genus Blautia (light grey) before and after treatment with the placebo (B);

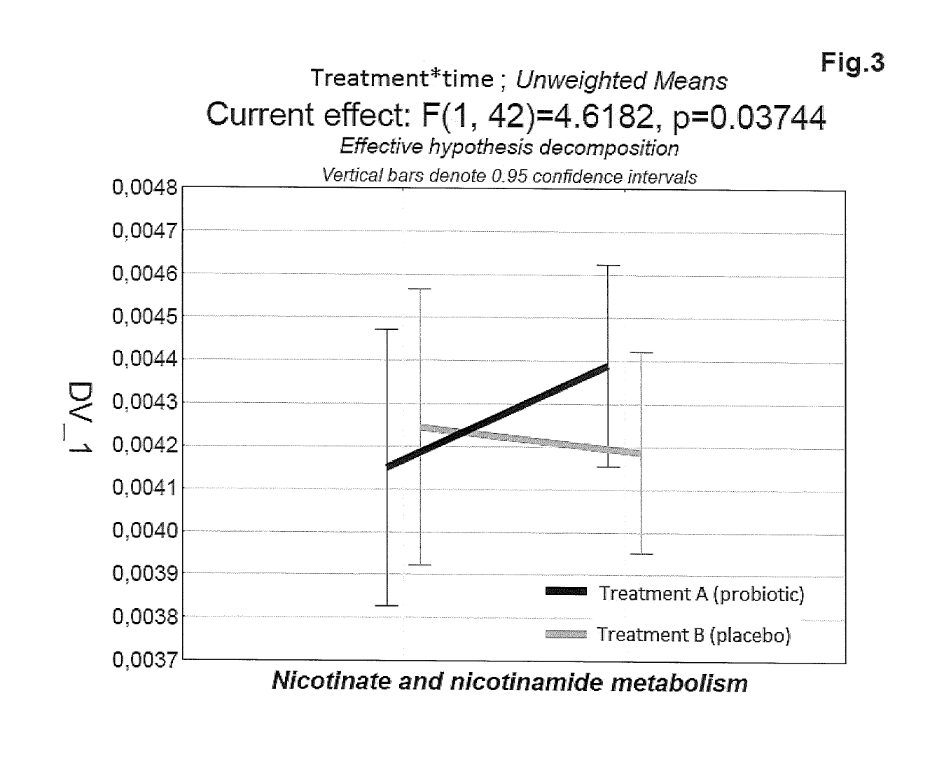

FIG. 3 shows the result of the statistical analysis conducted to establish the increase in the metabolism of nicotinic acid before and after treatment with the composition of the present invention and the decrease therein before and after treatment with the placebo; and

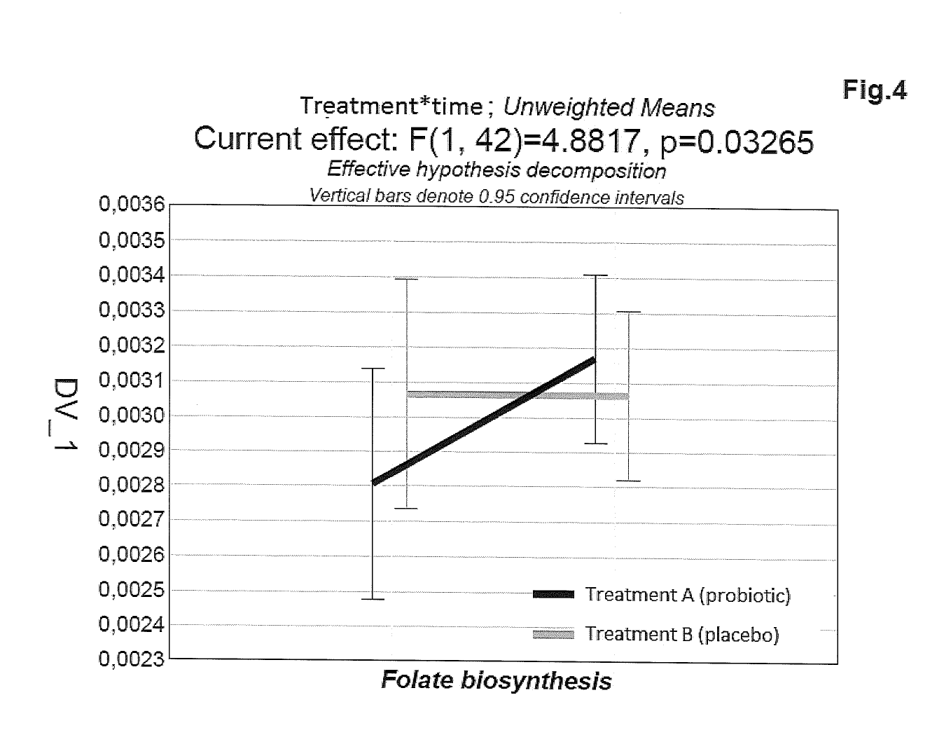

FIG. 4 shows the result of the statistical analysis conducted to establish the increase in the biosynthesis of folic acid before and after treatment with the composition of the present invention and an absence of any modifications, in contrast, before and after treatment with the placebo.

A first aspect of the present invention relates to a method for determining the change in the composition of the faecal microbiota of an individual following intake of a composition/formulation comprising microorganisms, according to a randomized, double-blind, placebo-controlled crossover protocol, said method comprising the steps of:
a) collecting information about the state of health and/or the eating habits of said individual before and/or during and/or after taking the composition or placebo according to a randomized, double-blind placebo-controlled crossover protocol;
b) obtaining at least one faecal sample from the individual before and/or during and/or after intake of the composition or placebo according to a randomized, double-blind placebo-controlled crossover protocol;
c) analyzing the microbiota by metagenomic analysis conducted on the faecal sample obtained in step b);
d) comparing, preferably qualitatively and/or quantitatively, the faecal microbiota of the individual before and/or during and/or after intake of the composition or placebo according to a randomized, double-blind, placebo-controlled crossover protocol.

In the context of the present invention, the term faecal microbiota means the whole of the populations of microorganisms which are present within the faeces of an individual and reflect the whole of the populations of microorganisms present in the intestine of the same. Therefore, the term faecal microbiota is meant here as a synonym of intestinal microbiota.

In particular, the microorganisms included in the composition of the present invention are bacteria and/or yeasts and/or other microorganisms, taken individually or in combination.

A composition comprising bacteria is particularly preferred for the purposes of the present invention. In particular, the bacteria belong to the genus selected from: Lactobacillus, Bifidobacterium, Bacillus, Propionibacterium, Streptococcus, Lactococcus, Aerococcus and Enterococcus. More preferably, said bacterium is of the genus Lactobacillus and/or Bifidobacterium.

In particular, the Lactobacillus is selected from: Lactobacillus paracasei, Lactobacillus acidophilus, Lactobacillus amylolyticus, Lactobacillus amylovorus, Lactobacillus alimentarius, Lactobacillus aviaries, Lactobacillus brevis, Lactobacillus buchneri, Lactobacillus casei, Lactobacillus cellobiosus, Lactobacillus coryniformis, Lactobacillus crispatus, Lactobacillus curvatus, Lactobacillus delbrueckii, Lactobacillus farciminis, Lactobacillus fermentum, Lactobacillus gallinarum, Lactobacillus gasseri, Lactobacillus helveticus, Lactobacillus hilgardii, Lactobacillus johnsonii, Lactobacillus kefiranofaciens, Lactobacillus kefiri, Lactobacillus mucosae, Lactobacillus panis, Lactobacillus collinoides, Lactobacillus paraplantarum, Lactobacillus pentosus, Lactobacillus plantarum, Lactobacillus pontis, Lactobacillus reuteri, Lactobacillus rhamnosus, Lactobacillus sakei, Lactobacillus salivarius and Lactobacillus sanfranciscensis.

Particularly preferred for the purposes of the present invention are bacteria belonging to the species Lactobacillus paracasei, more preferably the strain Lactobacillus paracasei DG.

The bacterial strain Lactobacillus paracasei DG was deposited by SOFAR S.p.A. with the National Collection of Microorganism Cultures of the Pasteur Institute in Paris on May 5, 1995, with the deposit number CNCM I-1572. Initially, the strain had the denomination of Lactobacillus casei DG sub. casei.

In particular, the bacteria of the genus Bifidobacterium are selected from: Bifidobacterium adolescentis, Bifidobacterium animalis, Bifidobacterium bifidum, Bifidobacterium breve and Bifidobacterium longum.

The yeasts are preferably of the genus Saccharomyces, more preferably of the species Saccharomyces cerevisiae.

In general, the microorganisms included in the composition of the present invention are individual microorganisms or combinations of any microbial species specified in the QPS list of the EFSA (http://www.efsa.europa.eu/it/search/doc/3020.pdf).

The microorganisms of the composition of the present invention are preferably live and the composition is thus also definable as a probiotic. Alternatively, the microorganisms of the composition are dead and/or in the form of a lysate or extract and hence the composition is also definable as a paraprobiotic. Therefore, the composition of the present invention is also a known or presumed probiotic or paraprobiotic.

In one embodiment of the invention, the composition comprises about 1-50 billion colony forming units (CFU) of microorganisms, preferably 15-30, more preferably 20-25 billion CFU of microorganisms.

In one embodiment of the present invention, the composition is formulated for oral administration. In particular, the composition is formulated in solid form, preferably as pills, capsules, tablets, granular powder, hard capsules, water-soluble granules, sachets or pellets.

Alternatively, the composition of the invention is formulated as a liquid, for example as a syrup or beverage, or else is added to a food, for example a yogurt, cheese, or fruit juice.

Alternatively, the composition of the invention is formulated in a form capable of exerting an action topically, for example as an enema.

In a further embodiment of the invention, the composition also comprises excipients generally accepted for the production of probiotic and/or pharmaceutical products.

In a further embodiment of the invention, the composition of the invention is enriched with vitamins, trace elements such as zinc and selenium, enzymes and/or prebiotic substances such as fructooligosaccharides (FOS), galactooligosaccharides (GOS), inulin, guar gum or combinations thereof.

As regards intake of the composition, as earlier explained, it follows a randomized, double-blind, placebo-controlled crossover protocol. In other words, during intake neither the investigator nor the individuals included in the trial are aware of the assigned treatments (the treatments are indistinguishable, double blind) and a same individual is exposed at different times to treatment both with the composition containing microorganisms and with the placebo (crossover), according to a random sequence.

In one embodiment of the invention, said protocol comprises the following phases: 1) a pre-recruitment phase, in which the individuals preferably do not take the composition comprising microorganisms or the placebo; and/or 2) a first treatment phase, in which the individuals preferably take the composition comprising microorganisms or the placebo; and/or 3) a wash-out phase, in which the individuals preferably do not take the composition comprising microorganisms or the placebo; and/or 4) a second treatment phase, in which the individuals preferably take the placebo or the composition comprising microorganisms.

Intake as per phase 2 and phase 4 takes place in a random, double-blind manner as specified above. It is clear that the individual who takes the placebo in the first phase will take the composition comprising microorganisms in the second phase and vice versa.

The duration of the different phase of the protocol is preferably the same. In particular, the duration of at least one of these phases, preferably of all the phases, is about four weeks.

In the context of the present invention, the term wash-out means a period falling between two phases of taking the composition comprising microorganisms or a placebo in which the individual does not take anything and should thus “expel” what he or she has taken previously, i.e. a period of absence of treatment aimed at eliminating every residual effect. In one embodiment of the invention, the composition of the present invention is taken preferably once a day, more preferably right after awakening.

Alternatively, taking it in the evening is also possible, preferably at least 3 hours after meals.

The step of collecting information regarding the state of health and/or the eating habits of the individual is preferably carried out by gathering said information in a questionnaire. Said questionnaire is prepared ad hoc to collect data regarding the state of health and/or the eating habits of an individual who implements the method of the invention.

In particular, said questionnaire is a standard sheet on which questions related to the state of health and/or the eating habits of said individual are formulated. As regards the state of health, the individual can respond using rating scales associated with each question. The rating scale is preferably a verbal numerical scale (VNS), or a visual analogue scale (VAS) or verbal rating scale (VRS). As regards eating habits, the individual can respond by indicating the foods he or she consumes daily, also specifying the amounts consumed where possible.

The step of collecting information is preferably carried out at the start of the pre-recruitment phase and/or before and/or after the first treatment phase and/or before and/or after the end of the second treatment.

The obtainment of at least one faecal sample preferably takes place at the start and/or at the end of the first treatment and/or at the start and/or at the end of the second treatment.

The faecal sample is preferably taken no earlier than 48 hours before, more preferably no earlier than 24 hours before being processed or stored at a temperature preferably comprised between +4° C. and −20° C., more preferably at −20° C., for a period that preferably does not exceed 7-10 days. Storage of the faecal sample before processing or storage at a low temperature preferably takes place at room temperature.

The step of analyzing the microbiota by metagenomic analysis of the faecal sample is carried out on the nucleic acids, preferably on the DNA extracted from the faecal microbiota.

In particular, the analysis of the microbiota by metagenomic analysis comprises at least one, and preferably all, of the following steps:
extracting the nucleic acids, preferably of the DNA from the faecal sample; and
molecularly typing the faecal microbiota.

Extraction of the nucleic acids in general, and DNA in particular, from the faecal sample is achieved using the procedures known to every person skilled in the art for that purpose.

In one embodiment of the invention, the typing of populations of microorganisms is achieved by analyzing the nucleotide sequence of at least one portion of the gene encoding a subunit of the ribosome, preferably the 16S subunit of the ribosome, i.e. the gene encoding the 16S rRNA molecule.

For this purpose, the DNA extracted from the faecal samples is amplified using techniques known in the art, for example by PCR. Preferably, the amplification is achieved by using a pair of oligonucleotides (primers); preferably by using SEQ ID NO: 1 (Probio_Uni 5′-CCTACGGGRSGCAGCAG-3′) and SEQ ID NO: 2 (Probio_Rev 5′-ATTACCGCGGCTGCT-3′) (Milani C, Hevia A, Foroni E, Duranti S, Turroni F, et al. (2013) Assessing the Fecal Microbiota: An Optimized Ion Torrent 16S rRNA Gene-Based Analysis Protocol. PLoS ONE 8(7): e68739).

The conditions for carrying out the PCR can vary depending on the quality and quantity of the nucleic acid it is desired to amplify and/or the primers used. In any case, setting the PCR conditions is a routine activity for every person skilled in the art.

Preferably, the portions of amplified nucleic acid are subsequently sequenced.

The person skilled in the art can use any known method for that purpose. Preferably, the methods used are selected from: sequencing based on the Sanger method, pyrosequencing methods and the Ion Torrent sequencing method.

In the case of Ion Torrent, it is preferable to use primers that preferably have adaptor sequences at the 5′ end. In the particularly preferred embodiment of the present invention, the adaptor sequences are SEQ ID NO: 1 and 2.

Once the sequences have been obtained and, therefore, once the populations of microorganisms of the faecal microbiota have been typed, the community of microorganisms is characterized, preferably by means of hierarchical clustering programs or taxonomic analysis and/or by constructing phylogenetic dendrograms, preferably with heat maps. To this end, QIIME software is particularly preferred for the purposes of the present invention.

Finally, the data obtained from the characterization analyses are preferably analyzed with statistical methods of a parametric and/or non-parametric type.

A further aspect of the present invention regards a kit for performing the method according to the present invention, said kit comprising:
an identification code of the kit;
at least one oral formulation of a composition comprising microorganisms, preferably belonging to the species Lactobacillus paracasei, more preferably the strain Lactobacillus paracasei DG, in an amount of between 1 and 50 billion colony forming units (CFU) of microorganisms, preferably 15-30, more preferably 20-25 billion CFU of microorganisms.
at least one oral formulation of a placebo not containing microorganisms; said composition of microorganisms being taken according to a randomized, double-blind crossover protocol controlled vis-à-vis said placebo, and said composition comprising microorganisms and said placebo being identified by a code.

The placebo is preferably identical in aesthetic appearance, i.e. in form, but differs in substance from said oral formulation of a composition comprising microorganisms, preferably belonging to the species Lactobacillus paracasei, more preferably the strain Lactobacillus paracasei DG. The oral formulation of the placebo contains no microorganisms.

In one preferred embodiment of the invention, the kit comprises at least 28 capsules or tablets or pills or buccal tablets or hard capsules or sachets containing the oral formulation of the composition comprising microorganisms, and, preferably, an equal number of tablets or pills or buccal tablets or hard capsules or sachets containing the oral formulation of placebo.

According to a preferred embodiment of the invention, said at least one oral formulation is at least one capsule, at least one tablet, at least one pill, at least one buccal tablet, at least one hard capsule, at least one sachet or at least one pellet.

Said oral formulations are identified by a code, for example a colour code, a numerical code, an alphabetic code, or an alphanumeric code. For the purpose of the method, said code will serve to understand when the composition comprising microorganisms has been taken and when the placebo as earlier described has been taken.

The oral formulations are identical in aesthetic appearance, i.e. in form, but differ in substance because one contains the composition comprising microorganisms and the other one contains a placebo. Moreover, the two formulations are each identified by a code.

In this manner, the composition comprising microorganisms and the placebo contained within a kit are such as to be indistinguishable by any individual. Moreover, the composition comprising microorganisms and the placebo contained in the kit are univocally identified by any code whatsoever, for example a colour code, a numerical code, an alphabetic code, or an alphanumeric code.

The correspondence of this code with the nature of the substance, i.e. whether it is the composition comprising microorganisms or the placebo, is known only to the producer of the kit.

According to a further embodiment of the present invention, the kit further comprises questionnaires prepared ad hoc for collecting data regarding the state of health of the individual who implements the method of the invention.

In particular, the questionnaires are standard sheets on which questions related to the state of health and/or the eating habits of said individual are formulated. As regards the state of health, the individual can respond using rating scales associated with each question. The rating scale is preferably a verbal numerical scale (VNS), or a visual analogue scale (VAS) or verbal rating scale (VRS). As regards eating habits, the individual can respond by indicating the foods he or she consumes daily, also specifying the amounts consumed where possible.

A further aspect of the present invention regards the use of said kit for diagnostic and/or therapeutic purposes.

### EXAMPLE

### Treatment

A randomized, double-blind, placebo-controlled crossover study of dietary intervention was conducted on healthy individuals.

Volunteers were recruited in accordance with the following criteria:
inclusion criteria: healthy men and women, ranging in age between 18 and 55 years; signing of informed consent form;
exclusion criteria: antibiotic treatment in the month preceding the examination; episodes of viral or bacterial enteritis in the 2 months preceding the first examination; gastric or duodenal ulcers in the 5 years preceding the first examination; pregnancy or breastfeeding; recent or presumed cases of alcoholism and drug intake; other conditions of non-compliance with the study protocol.

The probiotic dietary intervention was carried out in accordance with a crossover design, as schematized in Table I below.

TABLE I

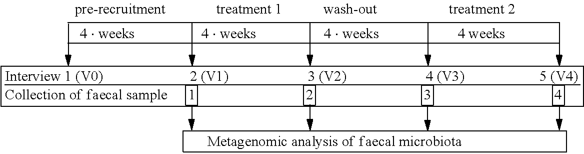

In the pre-enrolment step (4 weeks) the volunteers followed their usual diet, without consuming probiotic fermented milk products (traditional yogurt was thus permitted), probiotic dietary supplements, or prebiotic dietary supplements.

At the end of the pre-enrolment period, the volunteers were randomized to receive one capsule per day of a probiotic or placebo for 4 weeks.

By way of example, Enterolactis Plus was used as the probiotic to be administered; it consists in 420 mg capsules containing 24 billion CFU (colony forming units) of Lactobacillus paracasei, strain DG.

The placebo consisted in capsules identical in appearance to the probiotic ones, obviously devoid of the probiotic agent.

The flavour and colour of the active substance (i.e. the probiotic) and the placebo were identical.

The product was taken in the morning on an empty stomach, at least ten minutes before breakfast or, if forgotten, in the evening before going to bed and in any case at least two hours after the last meal.

After the first four weeks of treatment, the volunteers went through a four-week wash-out period identical to the pre-enrolment period.

At the end of the wash-out period, the volunteers took one capsule per day of Enterolactis Plus or placebo for four weeks in accordance with the crossover design described above.

In summary, the study involved 4 phases, each of which lasting 4 weeks:
Pre-recruitment phase: the individuals underwent neither treatment with Enterolactis Plus, nor treatment with the placebo.
Treatment 1: the individuals underwent treatment with Enterolactis Plus or treatment with the placebo.
Wash-out: the individuals underwent neither treatment with Enterolactis Plus, nor treatment with the placebo.
Treatment 2: the individuals underwent treatment with the placebo or treatment with Enterolactis Plus, respectively.

### Examinations and Sample Collection.

Each volunteer was initially instructed as to the entire procedure to be followed, which involved a total of 5 meetings per volunteer.

During the first meeting, informed consent was obtained along with the volunteer's personal data. The volunteer also received general information about how the study was to be carried out and was instructed about the changes in the diet to be applied in the subsequent 4 weeks of pre-enrolment (prohibition from consuming the previously specified products). After 4 weeks, the volunteer went to the second meeting with a faecal sample (sample T0), collected during the previous 24 hours in a special container handed over during the first meeting.

To ensure optimal preservation, the faecal samples were stored at room temperature and delivered to the laboratory within 24 hours.

During the second meeting, moreover, the volunteer was given the probiotic product (or placebo) to be taken during the next 4 weeks. Moreover, the volunteer was instructed as to how to take the product.

At the end of the 4 weeks of taking the product (or placebo), the volunteer went to the third meeting with another faecal sample (sample T1) collected during the previous 24 hours.

During the third meeting, the volunteer completed a questionnaire on the possible effects, both positive and undesirable ones, deriving from consumption of the product.

The volunteer was then instructed about the next 4 weeks, during which he or she again did not take the previously mentioned products.

At the end of these 4 weeks, the volunteer went to the fourth meeting with a faecal sample (sample T2) and received the probiotic product (or placebo) to be taken during the next 4 weeks.

Finally, after 4 weeks of taking the product (or placebo), the volunteer went to the fifth meeting to deliver the last faecal sample (sample T3).

During this last meeting, the volunteer completed a questionnaire analogous to the one received during the third meeting.

All the faecal samples collected were stored at −20° C. for no more than 7 days before being subjected to analysis of the microbiota.

### Analysis of Faecal Microbiota

The faecal microbiota was evaluated by analyzing the nucleotide sequence of portions of the gene encoding the 16S rRNA bacterial ribosomal subunit. More specifically, a metagenomic strategy was adopted; it consists in short in the following steps:
1. extracting, quantifying and normalizing the metagenomic DNA from the faecal samples;
2. amplifying the V3 hypervariable region of the bacterial gene encoding the 16S rRNA by PCR;
3. quantifying the PCR products;
4. sequencing the amplification products;
5. bioinformatically analyzing the sequences.

The procedures according to steps 1 and 3 are techniques that are well known in the art and they are thus performed with the protocols commonly used in this field. For example, the methods described in laboratory manuals such as those by Sambrook et al. 2001, or Ausubel et al. 1994. Step 2 of amplifying the V3 region of the 16S ribosomal RNA genes was performed by means of the DNA amplification technique known as PCR, using Probio_Uni 5′-CCTACGGGRSGCAGCAG-3′ (SEQ ID NO: 1) and Probio_Rev 5′-ATTACCGCGGCTGCT-3′ (SEQ ID NO: 2) as oligonucleotides (primers).

In particular, the pair of primers SEQ ID NO: 1 and 2 amplifies the V3 region of the 16S rRNA gene.

Step 4 can be performed with the techniques known in the art for this purpose, for example techniques based on the Sanger method, pyrosequencing or the Ion Torrent Fusion Primers sequencing method used in the specific example of the present invention according to the protocol described in the materials and methods section of the scientific article by Milani et al. (2013).

In the case of the Ion Torrent technique, the primers are designed and synthesized in such a way as to include, at the 5′ end, one of the two adaptor sequences used in this specific DNA sequencing technique. In this case, the adaptor sequences were SEQ ID NO: 1 and 2.

The conditions under which the PCR was performed are the following:
5 minutes at 95° C.;
30 seconds at 94° C., 30 seconds at 55° C., and 90 seconds at 72° C. for 35 cycles;
10 minutes at 72° C.

At the end of the PCR, the integrity of the amplificate was verified by electrophoresis.

Step 5 of the method, necessary for characterizing the microbial communities, can be carried out with numerous techniques presently known for this purpose. More specifically, use was made of: hierarchical clustering, taxonomic analysis and construction of phylogenetic dendrograms with heat maps according to the protocol described in the materials and methods section of the scientific article by Milani et al. (2013); more specifically, the analysis of sequence data was conducted using QIIME software.

### Statistical Analysis of the Data

The statistical analysis was conducted using STATISTICA software (Statsoft Inc., Tulsa, Okla., USA).

In order to reveal significant differences, the data were analyzed using both parametric (multivariate and univariate repeated-measures ANOVA) and non-parametric (Wald-Wolfowitz and Mann-Whitney) statistical methods.

The normality of the data series (important assumption for ANOVA) was evaluated by means of the Shapiro-Wilk and Kolmogorov-Smirnov tests.

### Results of the Treatment

The study was completed by a total of 22 individuals (11 females and 11 males).

Thirty-three individuals were initially enrolled, but 11 of them withdrew early for various reasons: intake of antibiotics (4), refusal to continue the study (1), frequent episodes of diarrhoea (1), intake of other probiotics during the study period (3), drastic change in eating habits (1), and seasonal influenza with episodes of diarrhoea (1).

Upon the conclusion of the study and completion of the analysis of the results of the two treatments, the blind was broken and it was seen that: treatment A is the active treatment, containing Lactobacillus paracasei DG; treatment B is the placebo, identical on the exterior to the active treatment, but devoid of lactobacilli.

When the data obtained from the study were analyzed, a high stability, from a taxonomic viewpoint, of the intestinal microbiota of the study participants was observed.

In fact, it was found that:
a) two bacterial divisions of the 15 identified, namely, Bacteroidetes and Firmicutes, constitute over 90% of the sequences;
b) 11 families of the 131 identified constitute over 90% of the sequences; and
c) 20 genera of the 262 identified constitute over 90% of the sequences.

Moreover, this study confirmed that human intestinal microbiota at lower taxonomic levels (i.e. at the family and genus levels) is highly variable from one individual to another.

Therefore, the experimental evidence demonstrated the necessity of conducting, on a healthy population, crossover intervention trials in order to prevent the marked inter-individual variability from hiding the possible effects of the probiotic treatment or leading to false statistical positives.

When the modifications induced in the intestinal microbiota by the two treatments were evaluated, a statistically significant difference emerged in terms of genera only in the group receiving the treatment with Lactobacillus paracasei DG (active treatment). More specifically, an increase in the genus Coprococcus was observed. In fact, as can be noted in FIGS. 1.1, 2.1 and 2.2, before and after treatment with Lactobacillus paracasei DG a statistically significant increase in coprococci was observed. In contrast, a moderate reduction thereof was seen in the group receiving the placebo treatment.

Moreover, after treatment with Lactobacillus paracasei DG, a statistically significant reduction in bacteria of the genus Blautia was observed. In contrast, a slight increase thereof was seen in the group receiving the placebo treatment (FIGS. 1.2, 2.1 and 2.2).

Coprococci are among the main producers of butyrate at the intestinal level.

Butyrate is a fundamental compound at the intestinal level, since on the one hand it contributes to restoring the functional integrity of the intestinal mucosa and maintaining it over time, and on the other hand it has important anti-inflammatory effects, so much so that it is used as an adjuvant to dietary treatments for intestinal colopathies (e.g. chronic inflammatory intestinal diseases).

Moreover, an analysis of their genome reveals that these bacteria can use succinate as a fermentation substrate.

This information is fundamental, in consideration of the fact that members of the genus Blautia generate acetate and succinate as main end products of the fermentation of glucose.

Succinate is considered an ulcerogenic factor, capable, therefore, of exacerbating the condition of individuals with ulcerative colitis, since it is probably to blame for the mucosal damage present above all in the active phases of the disease.

In conclusion, following treatment with a probiotic, in this case following the administration of Lactobacillus paracasei DG, one observes an increase in the bacteria belonging to the genus Coprococcus and hence an increase in the intestinal concentration of butyrate.

At the same time, one observes a reduction in the concentration of succinate, which may be to blame for mucosal damage in individuals with ulcerative colitis, in a direct manner, because following treatment with the probiotic, in this case following the administration of Lactobacillus paracasei DG, there is a reduction in the bacteria belonging to the genus Blautia, and, in an indirect manner, because the increased population of coprococci is further able to decrease the concentration of succinate by using it as a substrate in their fermentation process.

In conclusion, following treatment with the probiotic, in the specific example following the administration of Lactobacillus paracasei DG, there is an increase in the concentration of butyric acid in the faeces of individuals, with a simultaneous reduction in other organic acids, such as succinic acid.

The data relating to the composition of faecal microbiota were used, finally, in a bioinformatic analysis aimed at a virtual reconstruction of the metagenome based on knowledge of the bacterial genomes (Okuda S, Tsuchiya Y, Kiriyama C, Itoh M, Morisaki H. Virtual metagenome reconstruction from 16S rRNA gene sequences. Nat Commun. 2012; 3: 1203); in other words it was established in silico which potential genes are present and how abundantly in a given microbiota. This analysis made it possible to verify a putative increase in the encoding genes for the synthesis of folic acid and metabolism of nicotinic acid (FIGS. 3 and 4). These two molecules represent important vitamins for the human host (respectively named vitamin B9 and B3). Vitamin B9, in particular, represents a nutritional factor of primary importance, a deficiency of which, especially in specific physiological conditions such as pregnancy, can lead to serious health consequences. Treatment with the probiotic used in this study could therefore favour the ability of intestinal microbiota to produce folic acid (vitamin B9), with a consequent nutritional benefit for the human host.

1. An in vitro method for determining the change in the composition of the fecal microbiota of an individual, comprising
(i) obtaining a fecal sample from the individual;
(ii) administering (a) a composition comprising microorganisms, or (b) a placebo to the individual;
(iii) obtaining a second fecal sample from the individual after administration of the composition or placebo;
(iv) analyzing the microbiota in the fecal samples by performing a metagenomic analysis on the fecal samples;
(v) comparing, qualitatively and/or quantitatively, the fecal microbiota of the samples before and after taking the composition or placebo,
wherein a change in fecal microbiota indicates a reduction in the intestinal proliferation of pathogenic microorganisms, an increase in the integrity of the intestinal mucosa, and/or an increase in the ability to repair intestinal lesions.

2. The method according to claim 1, wherein said microorganisms are bacteria and/or yeasts taken individually or in combination.

3. The method according to claim 1, wherein said microorganisms belong to a genus selected from the group consisting of: Lactobacillus, Bifidobacterium, Bacillus, Propionibacterium, Streptococcus, Lactococcus, Aerococcus and Enterococcus.

4. The method according to claim 3, wherein said microorganisms are bacteria of the genus Lactobacillus.

5. The method according to claim 4, wherein said microorganisms are bacteria of the species Lactobacillus paracasei.

6. The method according to claim 1, wherein said microorganisms are present in the composition in an amount of between 1 and 50 billion colony forming units (CFU) of microorganisms.

7. The method according to claim 1, wherein said microorganisms are present in the composition as live or dead microorganisms, or in the form of a lysate or extract.

8. The method according to claim 1, wherein the composition comprising microorganisms is formulated for oral administration in solid form, in the form of pills, capsules, tablets, granular powder, hard capsules, water-soluble granules, sachets or pellets.

9. The method according to claim 1, wherein the metagenomic analysis comprises:
Extracting the nucleic acids from the fecal sample, and
Optionally, molecularly typing the microorganisms present in the fecal microbiota.

10. The method according to claim 9, wherein the typing of fecal microbiota is performed by analyzing the nucleotide sequence of at least a portion of the gene encoding the 16S subunit of the ribosome.

11. The method according to claim 9, wherein the typing of fecal microbiota is achieved by amplifying the nucleotide sequence of at least a portion of the gene encoding the 16S subunit of the ribosome by PCR.

12. The method according to claim 11, wherein the PCR is performed using SEQ ID NO: 1 and 2.

13. The method according to claim 12, wherein the amplified nucleotide sequence is sequenced using the technique of Ion Torrent sequencing.

14. The method according to claim 9, wherein the microorganisms are characterized by means of hierarchical clustering programs and/or taxonomic analysis, and/or by constructing phylogenetic dendrograms.

15. The method according to claim 14, wherein the results of the characterization are analyzed by using parametric and/or nonparametric statistical methods.

16. A kit for performing the method according to claim 1, comprising:
an identification code of the kit;
at least one oral formulation of a composition comprising microorganisms in an amount of between 1 and 50 billion colony forming units (CFU) of microorganisms; and
at least one oral formulation of a placebo not containing microorganisms;
wherein said composition comprising said microorganisms and placebo are identified by a code.

17. The method of claim 1, further comprising collecting information about the state of health and/or the eating habits of said individual before and/or during and/or after taking the composition or placebo.

18. The method of claim 1, wherein the method is used to conduct a randomized, double-blind, placebo-controlled crossover protocol.

19. The method of claim 4, wherein microorganisms are selected from a species selected from the group consisting of: Lactobacillus paracasei, Lactobacillus acidophilus, Lactobacillus amylolyticus, Lactobacillus amylovorus, Lactobacillus alimentarius, Lactobacillus aviaries, Lactobacillus brevis, Lactobacillus buchneri, Lactobacillus casei, Lactobacillus cellobiosus, Lactobacillus coryniformis, Lactobacillus crispatus, Lactobacillus curvatus, Lactobacillus delbrueckii, Lactobacillus farciminis, Lactobacillus fermentum, Lactobacillus gallinarum, Lactobacillus gasseri, Lactobacillus helveticus, Lactobacillus hilgardii, Lactobacillus johnsonii, Lactobacillus kefiranofaciens, Lactobacillus kefiri, Lactobacillus mucosae, Lactobacillus panis, Lactobacillus collinoides, Lactobacillus paraplantarum, Lactobacillus pentosus, Lactobacillus plantarum, Lactobacillus pontis, Lactobacillus reuteri, Lactobacillus rhamnosus, Lactobacillus sakei, Lactobacillus salivarius or Lactobacillus sanfranciscensis.

20. The method of claim 4, wherein the microorganisms are from the strain Lactobacillus paracasei DG.


SEQUENCE LISTING (2 sequences)

SEQ ID NO: 1 - DNA, 17 bp, Artificial Sequence
  Synthesized
cctacgggrsgcagcag

SEQ ID NO: 2 - DNA, 15 bp, Artificial Sequence
  Synthesized
attaccgcggctgct